(19) United States
(12) Patent Application Publication
(10) Pub. No.: US 2006/0233713 A1
(43) Pub. Date: Oct. 19, 2006

(54) Gadolinium particle-based MRI contrast agents

(75) Inventor: David Brent MacQueen, Half Moon Bay, CA (US)

Correspondence Address: RICHARD ARON OSMAN;SCIENCE AND TECHNOLOGY LAW GROUP, 242 AVE VISTA DEL OCEANO, SAN CLEMEMTE, CA, 92672, US

(73) Assignee: SRI International

(21) Appl. No.: 11/404,815

(22) Filed: Apr. 14, 2006

Related U.S. Application Data
(60) Provisional application No. 60/673,147, filed on Apr. 19, 2005.

(51) Int. Cl. A61K 49/00

(52) U.S. Cl. 424/9.34; 977/930

(57) ABSTRACT

Compositions comprising purified gadolinium-exchanged carboxylate-alumoxane nanoparticles in an aqueous solution are provided for use as MRI contrast agents. Targeted imaging is provided by nanoparticles comprising surface-bound antibodies specific for biomolecules present in a patient.

### CROSS-REFERENCE TO RELATED APPLICATION

Priority is claimed to U.S. provisional application No. 60/673,147 filed Apr. 19, 2005, incorporated herein by reference.

The invention was made in part with Government support under grant number EB001486 awarded by the Department of Health and Human Services. The Government has certain rights in this invention.

### BACKGROUND OF THE INVENTION

The field of the invention is particle-based MRI contrast agents.

The most commonly studied MRI contrast agents are complexes of Gd(III) chelated by a molecule of structure 1 or 2: 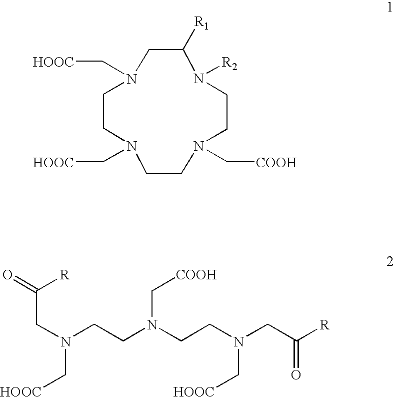
such as DO3A (R₁ and R₂═H); DOTA (R₁═H, R₂═COOH); MCTA (R₁═CH₃, R₂═COOH); HP-DO3A (R₁═H, R₂═CH₂CHOHCH₃); DTPA (R═OH); and DTPA-BMA (R═NHCH₃). A limitation of standard Gd(III) chelates is that in order for them to be useful as contrast agents in systems that involve targeting via antibodies, an estimated 10³ Gd ions per antibody molecule would be required to obtain observable signal enhancement (Lauffer, 1987). However, because only a limited number of functional groups can be conjugated to an antibody to avoid compromising its binding affinity, the contrast agent concentrations achieved by direct labeling of antibodies are typically not sufficient to generate detectable MRI contrast (Artemov, 2003). One approach used to overcome this limitation is to use larger carrier constructs that hold numerous Gd ions. For example, perfluorocarbon nanoparticles have been made with Gd(III) chelates and monoclonal antibodies bound at the surface (Lanza, 2003; Flacke, 2001). Gd(III) chelates and antibodies have also been attached to polylysine (Curtet, 1998).

Numerous other macromolecular agents have been investigated for their suitability as contrast agent cores including dextran, polyethylene glycol, dendrimers, etc. (reviewed in Kobayashi, 2005). Zeolite-enclosed gadolinium compounds (Young, 1995) and gadolinium endohedral metallofullerenes (Mikawa, 2001) are other examples of macromolecular contrast agents.

Boehmite is a hydrated form of alumina that has the nominal composition AlO₂H (see core structure 3) and exists in crystalline, pseudo-crystalline and amorphous morphologies.

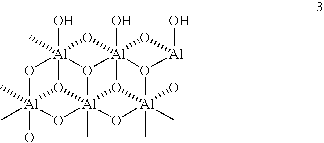

Alumoxanes (see core structure 4), which are also referred to as carboxylato- or carboxylate-alumoxanes, are derived from boehmite by attaching carboxylates to the boehmite surface via bonds between the oxygen of the carboxylate and the Al atoms of the boehmite, displacing hydroxyl groups from aluminum ions at the surface of the boehmite to form alumoxanes (Callender, 1997). Alumoxanes have been investigated for use in making water-processible ceramic slurries for slip casting (Callender, 1997). Kareiva et al (2001) describe a transmetalation reaction in which an alumoxane is mixed with a metal (M) acetonate (acac), resulting in the replacement of Al atoms at the surface of the alumoxane for other metal (M) ions (see scheme I). 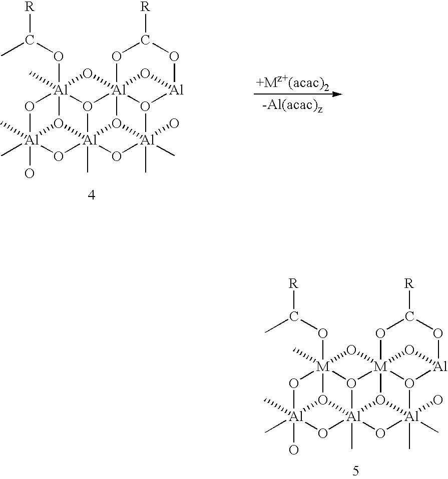
The resulting materials are used as intermediates in the preparation of mixed metal aluminum oxide materials, particularly for use in ceramics (Kareiva, 2001).

### BRIEF SUMMARY OF THE INVENTION

The invention relates to the discovery that purified gadolinium (Gd)-exchanged carboxylate-alumoxane nanoparticles have properties that make them suitable for use as MRI contrast agents. One aspect of the invention is a composition comprising purified Gd-exchanged carboxylate-alumoxane nanoparticles, preferably having an average particle size of less than 50 nm, in an aqueous solution. In one embodiment, at least 5% of surface metal atoms of the nanoparticles are gadolinium. In various embodiments, the nanoparticles exhibit a relaxivity greater than 6 mM⁻¹s⁻¹, and/or comprise less than 10 weight percent of the composition.

In specific embodiments, the carboxylate is selected from gluconate, 6-aminocaproate, a heterobifunctional polyethylene glycol, and a combination of gluconate and NH₂—PEG-COO⁻.

In various embodiments, the nanoparticles of the composition comprise surface-bound molecules such that when the composition is administered to a patient, the surface-bound molecules specifically bind biomolecules present in the patient. In specific embodiments, the surface-bound molecules are antibodies.

The compositions of the invention may be formulated for intravenous or oral administration.

Another aspect of the invention is a method of magnetic resonance imaging comprising the steps of: a) administering a composition comprising purified gadolinium-exchanged carboxylate-alumoxane nanoparticles to a patient; and b) obtaining a resultant enhanced magnetic resonance image of the patient.

Another aspect of the invention is a compound comprising a gadolinium chelate (e.g.

Gd-DOTA or GD-DTPA) linked to a nanoparticle, wherein the nanoparticle is a boehmite nanoparticle or a carboxylate-alumoxane nanoparticle.

### DETAILED DESCRIPTION OF THE INVENTION

The invention provides compositions and methods for contrast enhancement in magnetic resonance imaging (MRI). In one aspect, the subject composition comprises purified gadolinium (Gd)-exchanged carboxylate-alumoxane nanoparticles in an aqueous solution.

The nanoparticles are prepared from boehmite particles, which can be prepared by the hydrolysis of a trialkoxy aluminum compound (e.g. Al(sec-butoxide)₃) in water to form agglomerates that are subsequently peptized with aluminum nitrate (see e.g. Aivarias, 1996; and Callender, 1997). The boehmite particle size can be varied by varying the molar ratio of aluminum nitrate to Al(sec-butoxide)₃. For particle sizes of around 20-30 nm, the molar ratio of aluminum nitrate to Al(sec-butoxide)₃ is around 10:1 or higher. Lowering the concentration of aluminum nitrate results in larger particle sizes; molar ratios of less than about 4.5:1 aluminum nitrate to Al(sec-butoxide)₃, typically results in particles that are larger than preferred. The average size (i.e. diameter) of the boehmite particles is preferably less than 200, 100, or 50 nm. Most preferred is an average particle size of about 20-30 nm. In further embodiments, the maximum particle size does not exceed 200, 100, or 50 nm. Particle size can be determined using any suitable method such as with a particle size analyzer (e.g. LB-550 Dynamic Light Scattering Nanoparticle Size Analyzer, Horiba Ltd., Japan) by scanning electron microscopy (SEM), etc.

The boehmite particles are then reacted with carboxylic acids to form alumoxane using suitable methods (see e.g. Callender, 1997; Kareiva, 2001). The carboxylic acids can be aromatic or aliphatic, hydrophobic (e.g. octanoic acid, lauric acid, stearic acid, etc.) or hydrophilic (e.g. glycolic acid, hydroxybenzoic acids, 4-amino-n-butyric acid, etc.). Various other suitable carboxylic acids include n-hexanoic acid, dimethoxybenzoic acids, acetic acid, DOTA, DTPA, etc. In one preferred embodiment, the carboxylic acid used is gluconic acid to form gluconate-alumoxane. Gd-exchanged gluconate-alumoxane has very good water-dispersibility, allowing for maximum inner sphere exchange of water with the Gd ions on the surface of the nanoparticle, resulting in maximum relaxivity. The amount of surface coverage by the carboxylic acid can be calculated from knowing the particle size and morphology and measuring the weight loss observed by thermal gravimetric analysis (TGA). Typically, up to about one carboxylate ligand for every three surface aluminum ions can be bound to the particle.

In various embodiments the carboxylic acids have terminal functional groups that can be used to facilitate further surface modification of the nanoparticles. For example, amino acids such as glycine and alanine provide terminal amines that can be attached to additional molecules via amide linkage. A preferred amine-containing carboxylic acid is 6-aminocaproic acid to form 6-aminocaproate-alumoxane. Other preferred carboxylic acids include heterobifunctional polyethylene glycols (HB-PEGs) (Nektar Therapeutics, Birmingham, Ala.) that have carboxylic acid at one end of the PEG molecule and another functional group at the other end such as an amine, succinimide ester, maleimide, vinyl sulfone, thiol, etc. The HB-PEG typically has an average molecular weight of about 1000 to 6000, and preferably about 2,000-4,000.

Combinations of two or more carboxylic acids can be attached at the alumoxane surface. In one embodiment, a combination of gluconic acid and NH₂—PEG-COOH (Nektar Therapeutics, Huntsville, Ala.) are used, which results in Gd-exchanged carboxylate-alumoxane nanoparticles having very high relaxivity (see Table 1).

Next, transmetalation is performed using suitable methods (e.g. Cook, 1997; Harlan, 1997; and Kareiva, 2001) by reacting the alumoxane with Gd acetylacetonate (Gd(acac)₃) to replace aluminum (Al) atoms at or near the surface of the alumoxane with Gd atoms.

The final Gd concentration in the particles can be varied by varying the amount of Gd reagent used in the transmetalation reaction, and/or by varying the carboxylate moiety at the surface of the alumoxane (see Example 1). Preferably, at least 5% or 10% of the surface Al atoms are replaced with Gd atoms, and most preferably about 20-40% of the surface Al atoms are replaced with Gd atoms. For particles in the range of 20 nm to 50 nm the ratio of total Gd atoms to Al atoms in the particle is in the range of 1:10 to 1:1000000, preferably in the range of 1:1000 to 1:100000, and most preferably in the range of 1:1000 to 1:10000. For particles in the range of 50 nm to 100 nm the ratio of total Gd atoms to Al atoms in the particle is in the range of 1:100 to 1:10000000, preferably in the range of 1:1000 to 1:100000, and most preferably in the range of 1:1000 to 1:10000.

The nanoparticles can be further modified with additional surface molecules to impart the nanoparticles with desired properties, such as increased plasma half-life, preferential uptake by certain tissues, etc. Examples of such molecules include poly-1-lysine, polysaccharides such as polydextran, albumin, peptides, proteins, therapeutic agents (see e.g. Lanza, 2002), antibodies, nucleic acids, etc. In one embodiment, the nanoparticles comprise surface-bound molecules that specifically bind biomolecules present in a patient tissue that is desired to be imaged, resulting in site-specific accumulation of the contrast agent and enhanced contrast of the targeted tissue.

Suitable methods and applications of targeted nanoparticles for use in MRI are reviewed by Lanza et al (2003). Examples of surface molecule/second molecule binding pairs include ligand/receptor, complementary nucleic acids, avidin/biotin (see e.g. Artemov, 2003; Lanza, 2002), amyloidβ₁₋₄₀ peptide/amyloid beta (see e.g. Wadghiri, 2003), antibody/antigen (see e.g. Curtet, 1998; Anderson, 2000), etc. The surface molecules are readily attached to the nanoparticles using suitable methods. For example, thiol- or amine-reactive linking groups may be attached to the nanoparticles, to which the surface molecule is bound.

Antibodies can be readily attached to the nanoparticles using a variety of suitable methods (see e.g. Hampl, 2001; Pierce Applications Handbook and Catalog, 2003-2004 (Pierce Biotechnology, Rockford, Ill.); Hermanson, 1992; and Hermanson, 1996). For example, antibodies can be reacted with sodium metaperiodate to produce aldehyde moieties that can be directly conjugated to amines or hydrazides present on the alumoxane surface forming Schiff bases which are then stabilized by reduction with sodium cyanoborohydride. In another antibody attachment method, a dialdehyde, such as glycine or terephthaldicarboxaldehyde (TPDCA), is reacted with organic amines or hydrazides present on the alumoxane surface. The resulting Schiff base is reduced with cyanoborohydride, converting the surface to an aldehyde. The aldehyde is then reacted with an amine group on the antibody to again form a Schiff base which is reduced with sodium cyanoborohydride. The surface molecule can be an antibody that specifically binds the tissue desired to be imaged. Examples of various antibody targets include alpha (v)beta(3) integrin for targeting the neovasculature (Anderson, 2000), antibodies that specifically bind a tumor marker (e.g. carcinoembryonic antigen (CEA) for targeting colorectal carcinoma (Curtet, 1998)), tissue factor for targeting smooth muscle (Lanza, 2002), HER-2/neu receptor which is amplified in multiple cancers (Artemov, 2003), etc.

The nanoparticles are purified to remove any nitrate salts generated from the boehmite peptization, excess reagents (e.g. Gd(acac)₃) and by-products (e.g. Al(acac)₃) from the transmetalation reaction, and excess carboxylates and other surface modifying agents. Purification can be achieved by dialysis against purified water, or any other suitable purification method such as by size exclusion chromatography or centrifugation. The compositions contain purified nanoparticles when the Gd reagent (e.g. Gd(acac)₃) or the aluminum by-product (e.g. Al(acac)₃) used in the transmetalation reaction have been removed from the composition as determined by conventional Fourier transform infrared (FTIR) spectroscopy and H¹ NMR analysis.

The relaxivity of the nanoparticles may be characterized on a Minispec 60 MHz or similar relaxometer. Ti is measured, and the relaxivity (Rᵢ) is calculated according to the following equation: Rᵢ=(1/Tᵢ−1/Tᵢ control)×1/c, where Tᵢ is the relaxation time of the sample, Tᵢ control is the relaxation time of pure water, and c is the effective concentration of Gd in mM. The Gd and Al ion concentration of the nanoparticles can be determined by inductively coupled plasma atomic emission (ICP-AE) spectroscopy. In various preferred embodiments, the nanoparticles have relaxivity values greater than 6, 10, or 15 mM⁻¹s⁻¹.

The stability of the Gd in the nanoparticles can be determined using a dialysis cassette containing a known amount of nanoparticles suspended in a serum bath maintained at body temperature. Preferably, the Gd ions are not measurably leached from the nanoparticles after 24 hours. However, for compositions having very high relaxivity values, e.g. greater than 10 mM⁻¹s⁻¹, that are effective at lower doses, some Gd leaching is tolerable. In preferred embodiments, the composition leaches fewer than 5%, and more preferably fewer than 1% of its Gd ions when placed in 37° C. serum for 24 hours at a concentration of 1 mMol Gd/Kg serum.

The composition comprises, or consists essentially of, the purified nanoparticles suspended in an aqueous solution. To maintain nanoparticle size stability and prevent Oswald ripening, the concentration of the nanoparticles is preferably less than 6 weight percent, and more preferably 3 weight percent or less; though concentrations of up to about 10 weight percent can be achieved by adding dispersing agents such as Tween-20™ (Bio-Rad, Hercules Calif.) or Triton-X 100 (Dow Chemical, Midland, Mich.). The nanoparticle concentration can be determined inter alia by thermogravimetric analysis. The composition is pharmaceutically-acceptable and is typically administered to a mammalian patient orally or by intravenous injection. The composition may comprise additional additives commonly used in pharmaceutical formulations such as salts, electrolytes, dispersing agents, surfactants, preservatives, viscosity modifiers, flavorants, etc.

Another aspect of the invention is a compound comprising a Gd chelate linked to a nanoparticle, wherein the nanoparticle is a boehmite nanoparticle or a carboxylate-alumoxane nanoparticle. In this embodiment, boehmite or alumoxane nanoparticles are prepared as above, but transmetalation to replace aluminum atoms in the boehmite or alumoxane lattice with Gd atoms is not carried out. Rather, Gd is incorporated onto the surface of the alumoxane or boehmite nanoparticles by covalent linkage of the nanoparticles to a Gd chelate. In particular embodiments, the Gd chelate has the structure of structure 1 or 2 shown above. In specific embodiments, the Gd chelate is Gd-DOTA or GD-DTPA. In these embodiments, a carboxylic acid on the chelate is bound to the alumoxane surface. This can be accomplished by adding the chelate DOTA or DTPA to a boehmite particle to generate an alumoxane, and reacting the alumoxane with a soluble gadolinium salt (e.g. Gd(NO₃)₃) to generate the gadolinium-loaded alumoxane. Alternatively the chelated gadolinium can be added directly to boehmite to generate the gadolinium loaded alumoxanes.

Another aspect of the invention is a method of magnetic resonance imaging comprising the steps of: a) administering a composition of the invention to a patient; and b) obtaining a resultant enhanced magnetic resonance image of the patient. The compositions are typically administered intravenously or orally, but can also be administered by other suitable routes, e.g. intraperitoneally, directly into the tissue to be imaged, etc. The contrast agent is administered to the patient, and standard MRI imaging methods are used to obtain the resultant enhanced magnetic resonance image of the patient.

### EXAMPLE 1

### Preparation of Gd-exchanged Carboxylate Alumoxane Nanoparticles

The process to make the Gd-exchanged carboxylate-alumoxane (GadAl) nanoparticles starts with the preparation of a boehmite sol, its conversion to an alumoxane, and its transmetalation conversion to GadAl.

Boehmite particles were synthesized using previously described methods (Aivarias, 1996; and Callender, 1997) except that the boehmite precursor was prepared by the hydrolysis of Al(sec-butoxide)₃ as described by Yoldas (1975). 3.6 kg of water were heated to 85° C. with stirring, and 500 g of Al(sec-butoxide)₃ (Aldrich Chemical Co., Milwaukee, Wis. (Aldrich)) was added. After 5 minutes 51.2 g of Al(NO₃)₃ (Aldrich) was added to peptize the large agglomerates that initially formed into a well-dispersed colloid. Butanol that formed during the hydrolysis of the boehmite precursor was then distilled off from the mixture, and the mixture was heated at 85° C. for another 8 hours. The weight percent of boehmite was determined to be approximately 4-6% with particle sizes in the range of 15 to 60 nm as determined by a nanoparticle size analyzer (LB-55, Horiba Ltd., Japan).

Alumoxanes were prepared by reacting boehmite particles with carboxylic acids. One of the alumoxanes, gluconatoalumoxane, was prepared by mixing 1,063.4 grams of a sol containing 4.63 weight percent boehmite with 203.1 g of a 50% aqueous solution of gluconic acid (Aldrich) in a flask fitted with a condenser and heating to reflux for 24 hours with stirring. Excess gluconic acid was removed by dialysis. FTIR spectroscopy showed a shift in the band due to the carboxylic acid from 1730 cm⁻¹ to 1660 cm⁻¹, which indicates a metal-bound carboxylate functionality, and confirms that the alumoxane was generated.

Gd exchange was then carried out by reacting the gluconatoalumoxane with Gd acetylacetonate (Gd(acac)₃) (Aldrich) using previously described methods (Cook, 1997; Harlan, 1997; and Kareiva, 2001). 1001.4 g of a 3.72 weight percent gluconatoalumoxane aqueous sol was mixed with 5.55 g of Gd(acac)₃ in a flask fitted with a condenser and heated to reflux for 24 hours. The nanoparticles were purified by dialysis to remove Al(acac)₃ formed in the reaction and any excess Gd(acac)₃ and other impurities. Purity was confirmed by FTIR spectroscopy.

ICP-AE spectroscopy analysis indicated that the Gd ion concentration of the Gd-exchanged gluconatoalumoxane was 3.8×10⁻⁶ grams Gd/gram particles. We found that the Gd ion concentration varies with carboxylic acid used, and is typically in the range of about 2.0×10⁻⁶ to 9.0×10⁻⁶ g Gd/g particles. Considering that a 25 nm particle of boehmite contains roughly 10⁸ atoms, this translates to approximately 2,000 to 9,000 atoms per particle.

Relaxivity of the GadAl particles was determined by diluting stock preparations with Milli-Q water to 0.1 mMol/kg and 0.5 mMol/kg Gd. A 1.5T GE Signa MRI scanner with a standard quadrature head coil was used to estimate T₁ and T₂. T₁ relaxation was estimated from five spin-echo scans with varying repetition time (TR=150, 300, 600, 1200, and 2400 ms) at the minimum echo time (TE=14 ms). A minimization search routing was used to solve for T₁ and the fully relaxed signal intensity. T₂ relaxation was estimated from a two-echo, spin-echo acquisition with echo times of 20 and 80 ms at a 4 s repetition period. Results for some exemplary GadAl nanoparticles are presented in Table 1. The GadAl particles are designated by the carboxylic acid used for the surface modification. The FDA-approved Omniscan® (Gd-DTPA-BMA) was used as a control.

TABLE I

| | Gd (III) Conc. | r1 | r2 |
|---|---|---|---|
| Sample | (mMol/kg) | (mM⁻¹s⁻¹) | (mM⁻¹s⁻¹) |
| NH₂-PEG-COOH/Gluconic Acid | 0.1 | 34.9 | 79.6 |
| NH₂-PEG-COOH/Gluconic Acid | 0.5 | 37.8 | 95.9 |
| Gluconic Acid | 0.1 | 13.9 | 36.1 |
| Gluconic Acid | 0.5 | 12.9 | 34.4 |
| Aminocaproic Acid | 0.1 | 9.2 | 22.3 |
| Aminocaproic Acid | 0.5 | 12.1 | 30.4 |
| Omniscan ® | 0.1 | 2.3 | 3.5 |

The higher relaxivity values indicate that the GadAl analogs provide greater contrast enhancement compared to the control. In the mixed NH₂—PEG-COOH/Gluconic Acid which had the highest relaxivity values, our data indicate that the NH₂—PEG-COOH (MW=3400) accounted for 10% of the carboxylate surface modification (the remainder being gluconate).

### EXAMPLE 2

### Use of Gd-Exchanged Carboxylate Alumoxane Nanoparticles for Sentinel Node Imaging

Methodology for this study was adapted from previously described methods (Kobayashi, 2006). A mouse model of nodal metastases is used to show suitability of Gd-exchanged carboxylate-alumoxane (GadAl) nanoparticles for sentinel node visualization by MRI. Ten week-old female athymic nu/nu mice (NCI, Frederick, Md.) are used for all phases of the study.

Dynamic 3D-MR lymphangiography: GadAl nanoparticles are prepared as described in Example 1 having gluconate, aminocaproate or mixed gluconate/PEG-COOH surface carboxylate groups. Mice are anesthetized with 1.15 mg sodium pentobarbital i.p. administration. Thereafter, 0.10 mmol Gd/kg body weight of each GadAl contrast agent or 0.1 μmol Gd/kg body weight of Gd-[DTPA]-dimeglumine are injected subdermally into normal mammary glands or mammary tissue surrounding a tumor. The mice are wrapped with gauze to conserve their body temperature, and with an elastic tape around the chest to minimize the respiratory movement, and are placed at the center of the coils.

Dynamic MR images are obtained using a 1.5-Tesla superconductive magnet unit (Signa LX, General Electric Medical System, Milwaukee, Wis.) with a birdcage type coil of 3 cm diameter fixed by a custom-made coil holder. 3D-fast spoiled gradient echo (3D-fastSPGR, efgre3d package; Signa Horizon, GE) with chemical shift fat-suppression;

repetition time/echo time 19.2/7.2 ms; inversion time 47 ms; bandwidth 31.2 kHz, flip angle 30°, 4 excitations are obtained. The coronal images are reconstructed with 0.6-mm section thickness every 0.3-mm.

Correlating T1-weighted MRI signal intensity and concentration of Gd: Serum phantoms containing various concentrations of the GadAl agent with bovine serum are matched with MR images using the same imaging technique as described above. Three sets of phantoms, high (0.1-40 mM Gd/Kg body weight), intermediate (0.1-1 mM Gd/Kg body weight) and low (0.01-0.1 mM Gd/Kg body weight) concentrations are studied. A sample of Gd-[DTPA]-dimeglumine is used as an internal control.

In addition, the Gd(III) concentration is validated by calculating decreased T1 value in the axillary lymph node with the GadAl agent compared with the contra-lateral axillary lymph node. From the pre-injection imaging data, T1 values are calculated on both the right (control) and left axillary lymph nodes. Mice are then injected with 0.10 μmol Gd/Kg body weight of the GadAl agent into the left mammary gland. Then, consecutive MR images are taken at the maximum accumulation time point for the GadAL, with three different flip angles of 10°, 20°, and 30° using the same imaging protocol as described above. The concentration of Gd(III) is estimated in the left axillary lymph nodes based on T1 values.

Mouse Models: The GadAl nanoparticles are compared for visualization of lymphatics in non-tumor bearing mice. Thirty five non-tumor bearing athymic nu/nu mice are divided into seven groups (n=5). Mice are evaluated with 3D-fast spoiled gradient echo with 36 slice encoding steps; scan time 4 min 49 s at 6, 12, 18, 24, 30, and 36 min post-injection of each contrast agent. The slice data is processed into 3D images using the maximum intensity projection (MIP) method with the same window and level (window 3500 and level 2100) (Advantage Windows, General Electric Medical System).

The enhancement ratio is calculated by taking the average signal intensity in the axillary lymph node and dividing it by the signal intensity in the adjacent muscle.

The GadAl reagent exhibiting the highest contrast is selected for further testing in non-tumor bearing and tumor bearing mice. Non-tumor bearing athymic nu/nu mice (n=8) are anesthetized and injected with 0.10 mmol Gd/Kg body weight of the GadAl contrast agent into the mammary gland, and axial images are taken with serum phantoms containing two concentrations (0.10 and 0.5 mM Gd/Kg body weight) of GadAl using the 3D-fastSPGR using 16 slice encoding steps (scan time 2 min 25 s) 12, 24, 36, 48, and 60 min after injection. In order to calculate the concentration of Gd ions in the enhanced left axillary lymph node, linear correlations of signal intensity are made in comparison with phantoms.

To validate the concentration of GadAl agent, another method is used to calculate the Gd concentration in the enhanced left axillary lymph node. The consecutive MR images of 5 separate mice together with the same phantoms used above are taken with the same 3D-fSPGR protocol using 4 flip angles 10°, 20°, 30° and 40° at 24 min post-injection of 40 mM/20 μL GadAl agent in the left mammary gland. Signal intensity of the enhanced left axillary lymph node is corrected with that in the right axillary lymph node as a non-enhanced control. Then signal intensities obtained with different flip angles are plotted in order to calculate decreased T1 values in the left axillary lymph node by the existence of GadAl agent. Then Gd(III) concentrations are calculated based on these decreased T1 values in the enhanced node and the R1 relaxivity of GadAl agent at this condition. Technically, consecutive multi-flip angle scans are able to be performed once in 1 h because of the gradient driver overheat on the MRI system. Therefore, in order to calculate serial concentration of the GadAl agent, this separate set of mice is used only to obtain the T1 values. The serial concentrations of GadAl agent are calculated based on serial scan data under fixed flip angle (300) and obtained T1 values obtained from multiflip angle scans.

Seven micro-metastasis model mice are induced by injecting 107 PT-18 cells, into the left mammary pad in athymic nu/nu mice. Mice are imaged when tumors of 4-7 mm develop in the mammary pad usually at 15 days post-injection. Images are taken with 3D-fast spoiled gradient echo with 36 slice encoding steps; scan time 4 min 49 s at 6, 12, 18, 24, 30, and 36 min post injection of each contrast agent. All axillary lymph nodes are resected immediately after MRI scans and fixed with 10% formalin. Histology on a center slice of each sample is examined with hematoxylin and eosin (H-E) staining using a light microscope (×10-×400; BX51, Olympus, Melville, N.Y.).

Serial dynamic MR lymphangiograms of the PT-18 xenograft/lymph node micro-metastasis model mice ire obtained with 3D-fastSPGR 12, 24, 36, and 48 min after injection of the GadAl contrast agent (0.1 mMol Gd/Kg body weight). Calculation of Gd (III) concentration in left axillary lymph node in each mouse is performed with both phantom and T1 measurement methods as described above.

The foregoing examples and detailed description are offered by way of illustration and not by way of limitation. All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. As used herein, the singular forms “a,” “an,” and “the,” refer to both the singular as well as plural, unless the context clearly indicates otherwise.

### REFERENCES

Anderson et al, (2000) Magn Reson Med 44:433-9.

Aivarias et al, (1996) Chem. Mater. 8:2331-2340.

Artemov et al, (2003) Cancer Res. 63:2723-2727.

Cook et al, (1997) Proc of the 32^{nd} Intersoc. Energy Conv. Eng. Conf. 2:1210-1213.

Curtet et al, (1998) Invest. Radiol. 33:752-761.

Flacke et al, (2001) Circulation 104:1280-1285.

Hampl et al, (2001) Analytical Biochem. 288:176-187.

Harlan et al, (1997) Adv. Mater., 9:68-70.

Hermanson, G. T., Mallia, A. K., & Smith, P. K. (1992) Immobilized Affinity Ligand Techniques, Academic Press

Hermanson, Greg T. (1996) Bioconjugate Techniques, Academic Press

Kareiva et al, (2001) U.S. Pat. No. 6,207,130.

Kobayashi et al, (2006) J Control Release 111:343-51.

Kobayashi et al, (2005) Advanced Drug Delivery Reviews 57:2271-2286.

Lanza et al, (2002) Circulation 106:2842-7.

Lanza et al, (2003) MEDICAMUNDI 47:34-39.

Lauffer et al, (1987) Chem. Rev. 87:901-927.

Mikawa et al, (2001) Bioconjug. Chem. 12:510-514.

Unger et al, (1985) Invest. Radiol. 20:693-699.

Wadghiri et al, (2003) Magn Reson Med. 50:293-302.

Yoldas, (1 975) Ceramic Bulletin 54:289-290.

Young et al, (1995) J Magn Reson Imaging. 5:499-508.

What is claimed is:

1. A composition comprising purified gadolinium-exchanged carboxylate-alumoxane nanoparticles in an aqueous solution.

2. The composition of claim 1 wherein the nanoparticles have an average particle size of less than 50 nm.

3. The composition of claim 1 wherein the nanoparticles have an average particle size of 20 to 30 nm.

4. The composition of claim 1 wherein at least 5% of surface metal atoms of the nanoparticles are gadolinium.

5. The composition of claim 1 wherein the nanoparticles comprise less than 10 weight percent of the composition.

6. The composition of claim 1 wherein the carboxylate is selected from the group consisting of gluconate and 6-aminocaproate.

7. The composition of claim 1 wherein the carboxylate is a combination of gluconate and NH₂—PEG-COO⁻.

8. The composition of claim 1 wherein the carboxylate is a heterobifunctional polyethylene glycol.

9. The composition of claim 1 wherein the nanoparticles have a relaxivity greater than 6 mM⁻¹s⁻¹.

10. The composition of claim 1 wherein the nanoparticles comprise surface-bound molecules, wherein when the composition is administered to a patient the surface-bound molecules specifically bind biomolecules present in the patient.

11. The composition of claim 1 wherein the nanoparticles comprise surface-bound antibodies, wherein when the composition is administered to a patient the antibodies specifically bind biomolecules present in the patient.

12. The composition of claim 1 formulated for intravenous administration.

13. The composition of claim 1 formulated for oral administration.

14. A method of magnetic resonance imaging comprising the steps of:
a) administering the composition of claim 1 to a patient; and
b) obtaining a resultant enhanced magnetic resonance image of the patient.

15. The method of claim 14 wherein the nanoparticles have an average particle size of less than 50 nm.

16. The method of claim 14 wherein the nanoparticles comprise surface-bound molecules that specifically bind biomolecules present in the patient.

17. The method of claim 14 wherein the nanoparticles comprise surface-bound antibodies that specifically bind biomolecules present in the patient.

18. The method of claim 14 wherein the composition is administered intravenously.

19. A composition comprising a gadolinium chelate linked to a nanoparticle, wherein the nanoparticle is a boehmite nanoparticle or a carboxylate-alumoxane nanoparticle.

20. The composition of claim 19 wherein the gadolinium chelate is Gd-DOTA or GD-DTPA.
